(19) United States
(12) Patent Application Publication
(10) Pub. No.: US 2011/0052690 A1
(43) Pub. Date: Mar. 3, 2011

(54) PROCESS FOR MANUFACTURE OF A MEDICAMENT WITH GRANULATION AND PAN COATING

(76) Inventors: Avi Avramoff, Haifa (IL); Sima Volpert, Netanya (IL)

(21) Appl. No.: 12/935,808

(22) PCT Filed: Jun. 1, 2009
(86) PCT No.: PCT/IL09/00546

§ 371 Date: Nov. 19, 2010

Related U.S. Application Data
(60) Provisional application No. 61/129,041, filed on Jun. 2, 2008.

(51) Int. Cl. A61K 9/28

(52) U.S. Cl. 424/474; 264/129

(57) ABSTRACT

A process for the manufacture of a coated tablet comprising a serotonin-norepinephrine reuptake inhibitor (SNRI), such as venlafaxine. According to one embodiment of the present invention, there is provided a process for manufacturing a coated tablet containing a serotonin-norepinephrine reuptake inhibitor, the process comprising preparing a granulate of the serotonin-norepinephrine reuptake inhibitor using a low shear granulator; compressing the granulation into a core; and applying a coating to the core using a pan coater.

### FIELD OF THE INVENTION

The present invention relates to the field of pharmaceuticals, and more specifically to a process for the manufacture of a coated tablet comprising a serotonin-norepinephrine reuptake inhibitor, such as venlafaxine, using low shear granulation and pan coating.

### BACKGROUND OF THE INVENTION

Serotonin-norepinephrine reuptake inhibitors (SNRIs) are a class of antidepressant used in the treatment of clinical depression and other affective disorders. They are also sometimes used to treat anxiety disorders, obsessive-compulsive disorder, attention deficit hyperactivity disorder (ADHD) and chronic neuropathic pain.

SNRIs increase the levels of two neurotransmitters in the brain that are known to play an important part in mood, namely, serotonin and norepinephrine, by inhibiting their reabsorption into cells in the brain. These increased levels are believed to enhance neurotransmission, and thereby improve and elevate mood.

Currently available SNRIs include venlafaxine, desvenlafaxine, sibutramine, nefazodone, milnacipran, desipramine, duloxetine and bifadine.

Venlafaxine, 1-[(2-dimethylamino)-1-(4-methoxyphenyl)ethyl] cyclohexanol, is one of the most commonly used SNRIs for the treatment of depression. Venlafaxine and the acid addition salts thereof are disclosed in U.S. Pat. No. 4,535,186. Venlafaxine hydrochloride may be administered to adults in compressed tablet form in doses ranging from 75 to 350 mg/day, in divided doses two or three times a day.

It has been found that in therapeutic dosing with venlafaxine hydrochloride tablets, rapid dissolution results in a rapid increase in blood plasma levels of the active compound shortly after administration followed by a decrease in blood plasma levels over several hours as the active compound is eliminated or metabolized, until subtherapeutic plasma levels are approached after about twelve hours following administration, thus requiring additional dosing with the drug thereof. The most common side effect of such a plural daily dosing regime is nausea, experienced by about forty five percent of patients under treatment with venlafaxine hydrochloride. Vomiting also occurs in about seventeen percent of the patients.

The problem of frequent administration of venlafaxine may be solved by the use of extended release formulations, in which the tablet is provided with a coating which, once the tablet has been ingested, slowly releases the active ingredient into the gastrointestinal tract. The active ingredients may be released continuously, or in repeated small doses over time, usually for a period of 12 hours or more. The aim is to increase the time period during which a therapeutic drug concentration level in the blood is maintained.

Extended release formulations for oral administration of drugs are preferred for a number of reasons. For example, they enable the patient to ingest the formulation less frequently, which may lead to increased patient compliance with the dosing regimen. They may also result in fewer side effects, as peaks and troughs of the level of the drug in the bloodstream of the patient may both be decreased, leading to a more even drug level in the blood over a period of time. Such formulations may also provide a longer plateau concentration of the drug in the blood.

Many different types of extended release formulations are known in the art. Currently, sustained and controlled release drug delivery systems administered by the oral route are usually based on either a gel forming matrix or coated formulations, or combinations thereof. The selection of the proper type of such an extended release formulation is crucial for effective drug delivery which minimizes side effects, and hence for patient compliance.

U.S. Pat. No. 6,274,171, issued on Aug. 14, 2001, describes one attempted solution to the problem of frequent administration of venlafaxine. The disclosure teaches an extended release formulation, which features film-coated spheroids containing venlafaxine, which are placed in a hard gelatine capsule. However, spheroids are a more costly and less efficient solid dosage form to produce, and also require the additional procedural step of being placed in a hard gelatin capsule.

Tablets are a less costly and a more efficient solid dosage form to manufacture. An extended release tablet formulation for venlafaxine was first described in EP 1473030B1, which teaches a tablet comprising a core, over which an outer coating is layered.

The core is preferably prepared by granulation.

There are a number of granulation technologies available to pharmaceutical manufacturers, each having different strengths and weaknesses. These include:

i) Single pot granulation, in which a mixer/granulator dries granules in the same equipment without discharging. The granulation is done in a normal high shear processor; however, care must be taken to avoid the formation of lumps as they cannot be broken down before drying;

ii) Fluid bed top spray granulation, in which granulation is performed using fluid beds fitted with spray nozzles. iii) High shear granulation/fluid bed drying combination, which is the most commonly used industrial scale method for the production of pharmaceutical granules. One of the shortcomings of high shear granulation is the broad particle size distribution, which normally demands sizing by milling. The large sized compact lumps often found also give problems in the subsequent drying process;

iv) Continuous fluid bed granulation, in which material is introduced via a rotary inlet valve and discharged as granules by a second outlet valve. This technique is sometimes associated with problems in homogeneity of the granules;

and

v) Pellet production line, in which granules are produced from powders. This process cannot be carried out using a gastight design, which is problematic if the use of organic solvents is required.

The coating for an extended release tablet comprising an SNRI must be stable and strong enough to survive the handling of the tablet, and must not cause tablets to stick together during the coating process. The tablets should have a prescribed coating thickness on the surface of each tablet, with little inter- and intra-tablet variability, since the amount of coating controls the release profile and bioavailability of the drug. Large variations in coating thickness can adversely influence the effectiveness of the tablet.

Tablets have conventionally been coated using fluid bed equipment or pan coating. Water-insoluble film-forming polymers used in extended release coatings are generally thermodynamically unstable and tend to aggregate rapidly, resulting in clotting problems during spray-coating the drug-containing core. Fluid bed processors have been found to be problematic for applying aqueous films to large batches of tablets, since the low bed density and the relatively high mass of tablets requires large volumes of air to achieve adequate bed movement, but use of such large air volumes often creates violent tablet collisions in the coating zone, leading to damage to the substrate and applied coating. Furthermore, aqueous coatings require high temperatures to remove the water.

There is thus a widely recognized need for, and it would be highly advantageous to have, an improved process for the manufacture of an extended release coated tablet formulation for SNRIs, such as venlafaxine.

### SUMMARY OF THE INVENTION

According to one aspect of the present invention there is provided a process for manufacturing a coated tablet containing a serotonin-norepinephrine reuptake inhibitor, the process comprising preparing a granulate of the serotonin-norepinephrine reuptake inhibitor using a low shear granulator; compressing the granulate into a core; and applying a coating to the core using a pan coater.

According to a further aspect of the present invention, there is provided a coated tablet containing a serotonin-norepinephrine reuptake inhibitor, obtained by a process comprising preparing a granulate of the serotonin-norepinephrine reuptake inhibitor using a low shear granulator; compressing the granulate into a core; and applying a coating to the core using a pan coater.

According to some embodiments, the pan coater is a perforated pan coater.

According to some embodiments, the serotonin-norepinephrine reuptake inhibitor comprises one or more of venlafaxine, desvenlafaxine, sibutramine, nefazodone, milnacipran, desipramine, duloxetine and bifadine.

According to some embodiments, the coating provides an extended release profile for the serotonin-norepinephrine reuptake inhibitor in vivo.

According to some embodiments, the core further comprises a filler, such as, for example, microcrystalline cellulose. Optionally and preferably, the filler is present in an amount of at least about 40% w/w of the total formulation. Optionally and more preferably, the filler is present in an amount of from about 45% to about 65% weight per weight of the total formulation.

According to some embodiments, the core further comprises a water soluble cellulosic polymer and a water insoluble cellulosic polymer. Optionally, the soluble cellulosic polymer comprises at least one of hydroxypropyl methylcellulose, hydroxypropyl cellulose, hydroxyethyl cellulose, methyl cellulose, water-soluble carboxymethyl cellulose, and salts thereof, and combinations thereof. Preferably, the water soluble cellulosic polymer comprises hydroxypropyl methylcellulose, such as, for example high molecular weight hydroxypropyl methylcellulose. Optionally and preferably, thigh molecular weight hydroxymethyl cellulose has a viscosity of at least about 100 cps and/or a molecular weight of at least about 1,000,000 g/mol.

Optionally and preferably, the water soluble cellulosic polymer in the core is present in an amount of at least about 5% weight per weight of the total formulation, more preferably in an amount of from about 5% to about 20% weight per weight of the total formulation, and most preferably in an amount of from about 8% to about 16% weight per weight of the total formulation.

According to some embodiments, the water insoluble cellulosic polymer in the core is present in an amount of at least about 5% weight per weight of the total formulation, preferably in an amount of from about 5% to about 10% weight per weight of the total formulation.

According to some embodiments, the water insoluble cellulosic polymer in the core comprises cellulose acetate or ethyl cellulose, or a mixture thereof.

According to some embodiments, the coating comprises a water soluble cellulosic polymer and a water insoluble cellulosic polymer.

Optionally and preferably, the water soluble cellulosic polymer in the coating is present in an amount of up to about 5% weight per weight of the total formulation, preferably in an amount of from about 0.1% to about 3%, weight per weight of the total formulation, and more preferably in an amount of from about 0.3% to about 1%, weight per weight of the total formulation.

According to some embodiments, the water soluble cellulosic polymer in the coating comprises hydroxypropyl methylcellulose. Optionally, the hydroxypropyl methylcellulose in the coating comprises low molecular weight hydroxypropyl methylcellulose, such as, for example, that having a molecular weight of less than about 10,000 g/mol and/or a viscosity of less than about 10 cps.

According to some embodiments, the water insoluble cellulosic polymer in the core is present in an amount of up to about 15% weight per weight of the total formulation, more preferably in an amount of from about 2% to about 12% weight per weight of the total formulation.

Optionally and preferably, the water insoluble cellulosic polymer in the core comprises ethyl cellulose.

The present invention further provides a process for manufacturing a coated tablet containing venlafaxine, the process comprising preparing a granulate comprising venlafaxine or a pharmaceutically acceptable salt thereof; at least 40% of a filler, weight per weight of the entire formulation; at least 5% of a water soluble cellulosic polymer selected from the group consisting of hydroxypropyl methylcellulose, hydroxypropyl cellulose, hydroxyethyl cellulose, methyl cellulose, carboxymethyl cellulose, weight per weight of the entire formulation; and at least 5% of ethyl cellulose, weight per weight of the entire formulation; compressing the granulate into cores; and coating the cores with a mixture of a water soluble cellulosic polymer selected from the group consisting of hydroxypropyl methylcellulose, hydroxypropyl cellulose, hydroxyethyl cellulose, methyl cellulose, and carboxymethyl cellulose; wherein the granulate is prepared using a low shear granulator; and wherein the coating is applied using a perforated pan coater.

The present invention further provides a process for manufacturing a coated tablet containing venlafaxine, the process comprising preparing a granulate of venlafaxine, hydroxypropyl methylcellulose, microcrystalline cellulose and ethyl cellulose, wherein an amount of hydroxypropyl methylcellulose is greater than about 8% weight per weight of the tablet, an amount of microcrystalline cellulose is greater than about 40% weight per weight of the tablet, and an amount of ethyl cellulose is greater than about 5% weight per weight of the tablet; compressing the granulate into cores; and coating the cores with a mixture of aqueous ethyl cellulose and hydroxypropyl methylcellulose to obtain the coated tablets; wherein the granulate is prepared using a low shear granulator; and wherein the coating is applied using a perforated pan coater.

According to some embodiments, the core further comprises a lubricant, such as, for example, one or more of stearate salts, stearic acid, talc, castor oil, hydrogenated palm oil, starch, polyethylene glycol, sodium stearyl fumarate, compritol, waxes, or a combination thereof. Optionally and preferably, the lubricant comprises magnesium stearate. Optionally and preferably, the magnesium stearate is present in an amount of from about 0.25% to about 5% weight per weight of the core, more preferably in an amount of up to about 2% weight per weight of the core.

According to some embodiment, the core further comprises a plasticizer, such as, for example, one or more of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof. Optionally and preferably, the plasticizer comprises dibutyl sebacate. Optionally, the plasticizer further comprises polyethylene glycol. According to some embodiments, the plasticizer is present in an amount of up to about 5% weight per weight of the total formulation.

According to some embodiments of the present invention, there is provided a coated tablet prepared according to any of the above processes or according to any process described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

In case of conflict, the patent specification, including definitions, will control. In addition, the materials, methods, and examples are illustrative only and not intended to be limiting.

### DESCRIPTION OF THE PREFERRED EMBODIMENTS

The present invention is of a process for the manufacture of a coated tablet comprising a serotonin-norepinephrine reuptake inhibitor (SNRI), such as venlafaxine.

The present invention provides a number of advantages over background art methods. Firstly, the granulation process involves a single step, such that one vessel is used for the entire granulation process, including the drying and final mixing steps. Secondly, the method of the present invention enables a higher amount (up to 600 kg) of granulate to be processed, as compared to the methods of the background art. Thirdly, satisfactory homogeneity is obtained. Fourthly, the method requires a relatively small amount of granulation solution.

According to one embodiment of the present invention, there is provided a process for manufacturing a coated tablet containing a serotonin-norepinephrine reuptake inhibitor, the process comprising preparing a granulate of the serotonin-norepinephrine reuptake inhibitor using a low shear granulator; compressing the granulation into a core; and applying a coating to the core using a pan coater.

As used herein the term “core” is defined as an uncoated tablet.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As used herein the term “about” refers to ±10%.

For the production of oral dosage forms, high shear granulators are more commonly used than medium and low shear versions because their increased mechanical energy requires less granulation liquid, and tablets produced are considered to be more mechanically stable.

The present inventors have surprisingly found that low-shear granulation can be used to efficiently produce a stable, coated tablet formulation for an SNRI, such as venlafaxine.

A low shear granulator uses very little mechanical force to combine powders and binding solutions. As compared to high-shear methods, low-shear granulation requires cheaper equipment and produces a more porous granule, which dissolves more easily. The most commonly used low shear granulator is the fluid bed granulator, which uses a high volume of air flow to evaporate powders in a chamber, while a binding solution is sprayed onto the particles to form a light bond. A fluid bed granulator does not impart mechanical energy, but instead relies on the powder characteristics and the binding solution to form the powder into granules. The fluid bed granulator is not suitable for use with high viscosity binder solutions. The process of wet granulation involves three steps: blending, liquid binder addition, and wet massing or distribution of the liquid. After charging the powder to the mixer, a blending step is required to achieve a homogeneous blend.

The final stage of the wet granulation process is the liquid distribution or wet massing. This step can be compared with a kneading step during which the voids between granules are compressed and thereby the granules are densified.

The ribbon blender type mixer is very popular as a dry mixer. However, if small amounts of liquid are added or if a dry paste is formulated for the machine, the ribbon blender can serve as a very reliable granulator. This type of mixer is associated with a number of drawbacks. For example, a prolonged kneading time, which may result from material sticking to the walls of the granulating device, densifies the granules. This increases moisture exposure, particle size, wet paste appearance, and reduces porosity. The tendency of the material to stick to the side wall is pronounced in the ribbon blender.

Paddles instead of ribbons decrease sticking problems and the torque required. Paddle blenders as batch granulators can handle wetter paste. These paddle blenders are occasionally used as continuous granulators and have both lower torque and more applications than a continuous ribbon blender. The movement of the paste helps remove material from the paddles.

Two very popular ribbon or paddle blenders used as granulators are the topogranulator and the turbulizer.

The topogranulator is a batch-style ribbon blender granulator with the ability to either compress or mechanically fluidize the granulation. Compression while slightly wet increases the overall influence in the liquid on the particle size of the granulation.

The turbulizer is a continuous paddle granulator. By using continuous power feeders and liquid-metering pumps, the unit produces large quantities of product per hour in a very small space. The unit provides adjustable mixing, shear, and impact action based on the revolutions per minute (rpm) of the shaft and angle of the impact blade.

An alternative blender is the planetary mixer. The planetary motion of these granulators is created by rotating the agitator off an assembly in a direction opposite that of the rotation of the agitator assembly as it moves around the bowl. The planetary mixers are represented by many commercial names (e.g. Hobart, Kitchen Aide, Pony and AMF Glen granulators). All of these mixers have the same basic makeup, which includes (a) planetary motion, (b) removable bowl, and (c) top-drive agitators.

These mixers tend to be better at mixing dry powders in the horizontal plane than in the vertical. Lack of vertical mixing may require the materials to be dumped and readded to the bowl to obtain an acceptable dry mix.

Another mixer, blender, and granulator that has found application in the pharmaceutical industry is the Patterson-Kelly twin-shell liquid-solids blender. These twin-shell units are equipped with a jacket for heating and cooling, a vacuum take-off, and a liquid dispersion bar through which a liquid binder can be added. As the blender rotates, liquid is sprayed into the powder charge through the rotating liquid dispersion bar, located concentric to the trunnion axis. The bar's dog-eared blades, rotating at 3300 rpm, aerates the powder to increase the speed and thoroughness of the blend. Granulation can be controlled by the rate of binder addition through the dispersion bar. After heating, the liquid of the binder is removed under reduced pressure. Mixing, granulating, heating, cooling, and removal of excess liquid are carried out in a continuous operation in an enclosed system, thereby protecting the contents from contamination and the adjacent area from contamination by the contents. Once the granulation process is completed, the remaining excipients can be added and blended by the simple rotating action of the blender. This unit is also known as a liquid-solids processor.

The coating is preferably applied using pan coating. Typically, a batch of tablets is loaded into a pan, and the coating solution is applied to the tablets as the pan rotates in order to coat the tablets. The coating uniformity of the tablets is based on a number of variables such as the design of the pan, pan rotation speed, baffle design within the pan, number of tablets, tablet size, tablet shape, and atomization and distribution of the coating solution.

More preferably, the pan coater is a perforated pan coater (such as Glatt's pan coater and ACCELA COTA), which allows faster evaporation of water from the aqueous coating.

The process of the present invention may be used to produce a coated tablet comprising any SNRI inhibitor, such as, for example, venlafaxine, desvenlafaxine, sibutramine, nefazodone, milnacipran, desipramine, duloxetine or bifadine. Preferably, the SNRI inhibitor is venlafaxine.

The process of the present invention is optionally and preferably used to produce a tablet wherein the coating provides extended release of the SNRI inhibitor, such as the extended release tablet formulation for venlafaxine described in EP 1473030B1.

Hence, the process of the present invention is optionally and preferably used to produce an extended release coated tablet formulation for venlafaxine, wherein the core comprises venlafaxine and a filler, upon which core is disposed a coating comprising a mixture of a water insoluble cellulosic polymer and a water soluble cellulosic polymer, wherein the coated tablet is characterized as having a release profile of venlafaxine such that an extended release profile is obtained for venlafaxine in vivo.

Optionally and preferably, the filler is present in an amount of at least about 40% weight per weight of the total formulation. Unless otherwise noted, all percentages are given as percent weight per weight. By “total formulation”, it is meant the core and coating together.

Examples of suitable fillers include microcrystalline cellulose, sodium carboxymethycellulose, ethylcellulose, cellulose acetate, starch, lactose, glucose, fructose, sucrose, dicalcium phosphate, sorbitol, manitol, mantitol, lactitol, xylitol, isomalt, erythritol, and hydrogenated starch hydrolysates, or a mixture thereof.

Preferably, the filler comprises microcrystalline cellulose. More preferably, the filler solely comprises microcrystalline cellulose. Most preferably, microcrystalline cellulose is present in the core in a range of from about 45% to about 65% weight per weight of the total formulation.

Preferably, the core further comprises a water soluble cellulosic polymer and a water insoluble cellulosic polymer. The combination of water soluble and water insoluble cellulosic polymers for both the core and the coating provides the desired bioavailability and extended release profile.

More preferably, the water soluble cellulosic polymer in the core is present in an amount of at least about 5% weight per weight of the total formulation. Optionally and preferably, the water soluble cellulosic polymer in the core is present in an amount of from about 5 to about 20%, weight per weight of the total formulation. More preferably, the water soluble cellulosic polymer in the core is present in an amount of from about 8% to about 16%, weight per weight of the total formulation.

More preferably, the water soluble cellulosic polymer in the core comprises HPMC (hydroxypropyl methylcellulose). Optionally and more preferably, HPMC comprises a high molecular weight form of this polymer. By “high molecular weight”, it is meant a form of HPMC having a viscosity of at least about 0,1 Pascal second (100 cps), and/or a form of HPMC having a molecular weight of at least about 1,000,000 g/mol. One non-limiting example of such a high molecular form of HPMC is Methocel K100M^{λ,,¢} (Colorcon Inc., USA). Most preferably, the water soluble cellulosic polymer in the core is present in an amount of from about 8% to about 16%, weight per weight of the total formulation.

Alternatively or additionally, the water soluble cellulosic polymer may comprise one or more of HPMC (hydroxypropyl methylcellulose), hydroxypropyl cellulose (more preferably of the high viscosity type), hydroxyethyl cellulose, methyl cellulose, water-soluble carboxymethyl cellulose, salts thereof, and mixture thereof.

Preferably, the water insoluble cellulosic polymer in the core is present in an amount of at least about 5% weight per weight of the total formulation. More preferably, the water insoluble cellulosic polymer comprises ethyl cellulose. Most preferably, the water insoluble cellulosic polymer in the core is present in an amount of from about 5% to about 10%, weight per weight of the total formulation.

Alternatively or additionally, the water insoluble cellulosic polymer may comprise one or more of cellulose acetate and ethyl cellulose.

According to preferred embodiments of the present invention, an extended release coating is provided, comprising a water soluble cellulosic polymer and a water insoluble cellulosic polymer.

Optionally and preferably, the water soluble cellulosic polymer in the coating is present in an amount of up to about 5% weight per weight of the total formulation. More preferably, the water soluble cellulosic polymer in the coating is present in an amount of from about 0.1% to about 3%, weight per weight of the total formulation, and most preferably from about 0.3% to about 1%, weight per weight of the total formulation.

More preferably, the water soluble cellulosic polymer comprises HPMC (hydroxypropyl methylcellulose). Optionally and more preferably, HPMC comprises a low molecular weight form of this polymer. By “low molecular weight” form of HPMC, it is meant a polymer preferably having a viscosity of less than about 10 cps, and more preferably less than about 0.005 Pascal second (5 cps) and/or a polymer having a molecular weight of less than about 10,000 g/mol. One non-limiting example of such a low molecular form of HPMC is Methocel E5^{λ,,¢} (Colorcon Inc., USA).

Preferably, the water insoluble cellulosic polymer in the coating is present in an amount of up to about 15% weight per weight of the total formulation. More preferably, the water insoluble cellulosic polymer comprises ethyl cellulose. Most preferably, the water insoluble cellulosic polymer in the coating is present in an amount of from about 2% to about 12%, weight per weight of the total formulation. Also most preferably, ethyl cellulose in the coating is present in a range of from about 2 to about 12%, weight per weight of the entire formulation.

According to another embodiment of the present invention, there is provided a process for manufacturing a coated tablet containing venlafaxine, the process comprising preparing a granulate comprising venlafaxine or a pharmaceutically acceptable salt thereof; at least 40% of a filler, weight per weight of the entire formulation; at least 5% of a water soluble cellulosic polymer selected from the group consisting of hydroxypropyl methylcellulose, hydroxypropyl cellulose, hydroxyethyl cellulose, methyl cellulose, carboxymethyl cellulose, weight per weight of the entire formulation; and at least 5% of ethyl cellulose, weight per weight of the entire formulation; compressing the granulate into cores; and coating the cores with a mixture of a water soluble cellulosic polymer selected from the group consisting of hydroxypropyl methylcellulose, hydroxypropyl cellulose, hydroxyethyl cellulose, methyl cellulose, and carboxymethyl cellulose; wherein the granulate is prepared using a low shear granulator; and wherein the coating is applied using a perforated pan coater.

According to yet another embodiment of the present invention, there is provided a process for manufacturing a coated tablet containing venlafaxine, the process comprising preparing a granulate of venlafaxine, hydroxypropyl methylcellulose, microcrystalline cellulose and ethyl cellulose, wherein an amount of hydroxypropyl methylcellulose is greater than about 8% weight per weight of the tablet, an amount of microcrystalline cellulose is greater than about 40% weight per weight of the tablet, and an amount of ethyl cellulose is greater than about 5% weight per weight of the tablet; compressing the granulate into cores; and coating the cores with a mixture of aqueous ethyl cellulose and hydroxypropyl methylcellulose to obtain the coated tablets; wherein the granulate is prepared using a low shear granulator; and wherein the coating is applied using a perforated pan coater.

According to any of the embodiments of the present invention, the process optionally further comprises the step of adding a lubricant to the granulate. The lubricant may optionally be selected from the group consisting of stearate salts (magnesium, calcium, etc); stearic acid, talc, castor oil, hydrogenated palm oil, some type of starch, polyethylene glycol, sodium stearyl fumarate, compritol (glycerol behenate), waxes, or a combination thereof. More preferably, the lubricant comprises magnesium stearate, which most preferably is present in an amount of up to about 2% weight per weight of the core, although optionally a concentration of from about 0.25% to about 5% weight per weight may be used.

According to any of the embodiments of the present invention, the core optionally and more preferably further comprises a flow regulating agent. Preferably, the flow regulating agent includes at least one of colloidal silicon dioxide, talc, corn starch, dimethicone, and aluminum silicate. More preferably, the flow regulating agent comprises colloidal silicon dioxide, most preferably in an amount of up to about 1%, weight per weight of the total formulation.

According to other preferred embodiments of the present invention, the coating preferably further comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof. More preferably, the plasticizer comprises dibutyl sebacate, particularly for combination or use with ethyl cellulose. Most preferably, the plasticizer also comprises polyethylene glycol, of which a non-limiting example is Macrogol 400^{λ,,¢} (Uniqema, USA), particularly for use or combination with HPMC. Most preferably, the plasticizer is present in an amount of up to about 5%, which is most preferably in a range of from about 0.01% to about 3% of the total formulation, percent weight per weight.

For manufacture of the core, the water insoluble cellulosic polymer is dissolved in a suitable organic solvent such as ethyl alcohol for example, to form a granulation solution. The SNRI, water soluble cellulosic polymer, and filler, are then mixed. The water insoluble cellulosic polymer is then added to the mixture to form a granulate, using low shear granulation conditions. The granulate is dried, for example with a fluid bed dryer. The dried granulate is then milled, and then optionally blended to form a blend.

Optionally, a flow regulating agent and lubricant are sieved and mixed with the previously prepared blend. The mixture is then compressed to form the tablets.

For the coating, the water soluble cellulosic polymer and plasticizer are dissolved in water. The solution is then added to a suspension of water insoluble cellulosic polymer with the second plasticizer and stirred to form the coating solution.

The previously prepared cores are then coated with the coating solution, using a perforated coating pan.

In perforated coating pan systems, as the material inside is coated it increases in size and weight. Generally the materials to be coated accumulate adjacent an end wall and along a side wall of the drum in the system. As the drum rotates, the material is tumbled and is coated with a coating composition from one or more spray nozzles. Initially the material may form a mass and as the material is sprayed and increased in size the large particles migrate away from the end wall and cannot penetrate the mass of smaller particle adjacent the end wall. Eventually, substantially all of the material is uniformly coated such that the material forms a new mass wherein the particles are slightly larger than the original mass formed by the uncoated particles. The process repeats itself such that the particles are coated with additional composition from the spray nozzle, thereby again increasing in size and weight and migrating away form the end wall. The cycle continues until the particle achieve a desired uniform size.

An example of a perforated coating pan system is available under the Accela Cota brand sold by Thomas Engineering Incorporated, 575 West Central Road, Hoffman Estates, Ill. 60195-0198, U.S.A. Various size pans may be satisfactorily employed herein and include without limitation 15, 24, 48 and 60 inch pans, if desired. The size of the pan and dryer are not critical. The Compu Lab model sold under the Accela Cota brand works well for laboratory size charge (feed) quantities. Those of skill in the art will recognize that various size pans may be employed depending on the amount of materials to be coated and other coating operations.

The Accela Cota brand side perforated coating pan system comprises a rotating drum and as the drum is rotated containing the tablets to be coated, the coating composition is applied to the tablets by means of one or more nozzles positioned within the rotating drum so as to direct the coating composition to the tablets in the bed. As the pan is rotated and the coating composition is further applied to the tablets, the tablets achieve a desired coating. This apparatus is also a dryer for substantially drying the tablets as the tablets are coated. The side wall of the drum is perforated and a flow of air is provided into the drum through apertures for drying the coating composition on the tablets. A system is also provided on the apparatus for removing the outlet air and for removing the coated tablets.

The nozzles of this side perforated coating system are preferably adjustable and may be positioned nearer to and closer to the bed of tablets to be coated depending on the conditions of use and the desired coating composition quality and quantity, among other factors. Those of skill in the art will recognize that the distance of the nozzle or nozzles from the bed is important and may be adjusted to provide optimum coating compositions. In operation such nozzle placement distances will be an effective distance and will be selected from a plurality of available positions and will depend on the tablets being coated, the coating compositions, the degree of coating desired and other conditions of the particular coating operation, among other factors.

Those of skill in the art will recognize that one or more nozzles may be employed as desired to provide optimum coating. The number of nozzles is not critical and may be varied as needed depending on the coating operation and other factors. The nozzle throat diameter is typically from about 0.028 inch to about 0.100 inch although, greater and smaller throat diameters may be employed. A nozzle throat diameter of somewhere about 0.040 inch is preferred although that size is not critical. The nozzle(s) is preferably aimed perpendicularly or nearly perpendicular to the bed although other direction(s) of aim may be employed if desired. Those of skill in the art will recognize that the pan may be rotated at a speed selected from a plurality of operating speeds. The pan may be stopped after the material has been coated and the matter removed.

In general, an effective nozzle distance for applying a coating to a tablet using a side perforated pan coating system is in the range from being positioned less than about a ¼ inch from the bed to about 15 inches and preferably from about 8 to about 12 inches although greater of lesser nozzle distances may be employed if desired depending on the weight of tablets charged into the pan and coating system composition and other factors.

If desired, the same or a similar coating application system can be employed for both a first and a second or sequential coating applications or different coating application systems may be employed for a first or second or more coating applications. If desired, the same coating application system can be used to apply a first and second or more coatings with or without removal of the tablets from such a system between the first and second or more coatings.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above description, illustrate the invention in a non limiting fashion.

### Example 1

Venlafaxine HCl, microcrystalline cellulose (MCC), hydroxypropyl methyl cellulose (HPMC) and ethocel are mixed in a single pot device, for example a V-processor. Alcohol is sprayed onto the mixture in order to achieve a granulate. The granulate is dried in the same equipment. The dried granulate is then milled and blended to form a blend. Colloidal silicon dioxide and magnesium stearate are then sieved. The sieved materials are preferably mixed with the previously prepared blend. The mixture is then compressed to form tablets.

For the coating, polyethylene glycol (PEG) and HPMC are dissolved in water to form a solution. The solution is then added to a 30% aqueous dispersion of ethylcellulose with dibutyl sebacate and stirred for about 45 minutes to form the coating solution. The coating process is then performed in a perforated pan coater,

### Example 2

For manufacture of the core, ethyl cellulose is dissolved in ethyl alcohol for example, to form a granulation solution. Venlafaxine hydrochloride, HPMC, ethylcellulose and microcrystalline cellulose are then mixed under low shear conditions. Alcohol is then added to the mixture to form a granulate. The granulate is dried, for example with a fluid bed dryer. The dried granulate is then milled, and then optionally blended to form a blend.

Next, colloidal silicon dioxide and magnesium stearate are sieved. The sieved materials are preferably mixed with the previously prepared blend. The mixture is then compressed to form the tablets.

For the coating, PEG and HPMC are dissolved in water to form a solution. The solution is then added to and aqueous dispersion of 30% ethylcellulose with dibutyl sebacate and stirred for about 45 minutes to form the coating solution. The coating process is then performed in a perforated pan coater.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A process for manufacturing a coated tablet containing a serotonin-norepinephrine reuptake inhibitor, the process comprising:
preparing a granulate of the serotonin-norepinephrine reuptake inhibitor using a low shear granulator;
compressing said granulate into a core; and
applying a coating to said core using a pan coater.

2. The process of claim 1, wherein said pan coater is a perforated pan coater.

3. The process of claim 1, wherein said serotonin-norepinephrine reuptake inhibitor is selected from the group consisting of venlafaxine, desvenlafaxine, sibutramine, nefazodone, milnacipran, desipramine, duloxetine and bifadine.

4. The process of claim 3, wherein said coating provides an extended release profile for the serotonin-norepinephrine reuptake inhibitor in vivo.

5. The process of claim 4, wherein said core further comprises a filler.

6. The process of claim 5, wherein said core further comprises a water soluble cellulosic polymer and a water insoluble cellulosic polymer.

7. The process of claim 5, wherein said filler is present in an amount of at least about 40% w/w of the total formulation.

8. The process of claim 7, wherein said filler is present in an amount of from about 45% to about 65% weight per weight of the total formulation.

9. The process of claim 5, wherein said filler comprises microcrystalline cellulose.

10. The process of claim 6, wherein said water soluble cellulosic polymer in said core is selected from the group consisting of hydroxypropyl methylcellulose, hydroxypropyl cellulose, hydroxyethyl cellulose, methyl cellulose, water-soluble carboxymethyl cellulose, and salts thereof, and combinations thereof.

11. The process of claim 10, wherein said water soluble cellulosic polymer in said core comprises hydroxypropyl methylcellulose.

12. The process of claim 6, wherein said water soluble cellulosic polymer in said core is present in an amount of at least about 5% weight per weight of the total formulation.

13. The process of claim 12, wherein said water soluble cellulosic polymer in said core is present in an amount of from about 5% to about 20% weight per weight of the total formulation.

14. The process of claim 11, wherein said hydroxypropyl methylcellulose comprises high molecular weight hydroxypropyl methylcellulose.

15. The process of claim 14, wherein said high molecular weight hydroxymethyl cellulose has a viscosity of at least about 100 cps.

16. The process of claim 6, wherein said water soluble cellulosic polymer in said core is present in an amount of from about 8% to about 16% weight per weight of the total formulation.

17. The process of claim 6, wherein said water insoluble cellulosic polymer in said core is present in an amount of at least about 5% weight per weight of the total formulation.

18. The process of claim 17, wherein said water insoluble cellulosic polymer in said core is present in an amount of from about 5% to about 10% weight per weight of the total formulation.

19. The process of claim 6, wherein said water insoluble cellulosic polymer in said core is selected from the group consisting of cellulose acetate and ethyl cellulose, or a mixture thereof.

20. The process of claim 4, wherein said coating comprises a water soluble cellulosic polymer and a water insoluble cellulosic polymer.

21. The process of claim 20, wherein said water soluble cellulosic polymer in said coating is present in an amount of up to about 5% weight per weight of the total formulation.

22. The process of claim 21, wherein said water soluble cellulosic polymer in said coating is present in an amount of from about 0.1% to about 3%, weight per weight of the total formulation.

23. The process of claim 22, wherein said water soluble cellulosic polymer in said coating is present in an amount of from about 0.3% to about 1%, weight per weight of the total formulation.

24. The process of claim 20, wherein said water soluble cellulosic polymer comprises hydroxypropyl methylcellulose.

25. The process of claim 24, wherein said hydroxypropyl methylcellulose comprises low molecular weight hydroxypropyl methylcellulose.

26. The process of claim 25, wherein said low molecular weight hydroxypropyl methylcellulose has a molecular weight of less than about 10,000 g/mol and/or a viscosity of less than about 10 cps.

27. The process of claim 20, wherein said water insoluble cellulosic polymer in said core is present in an amount of up to about 15% weight per weight of the total formulation.

28. The process of claim 27, wherein said water insoluble cellulosic polymer in said core is present in an amount of from about 2% to about 12% weight per weight of the total formulation.

29. The process of claim 20, wherein said water insoluble cellulosic polymer comprises ethyl cellulose.

30. A process for manufacturing a coated tablet containing venlafaxine, the process comprising:
preparing a granulate comprising venlafaxine or a pharmaceutically acceptable salt thereof; at least 40% of a filler, weight per weight of the entire formulation; at least 5% of a water soluble cellulosic polymer selected from the group consisting of hydroxypropyl methylcellulose, hydroxypropyl cellulose, hydroxyethyl cellulose, methyl cellulose, carboxymethyl cellulose, weight per weight of the entire formulation; and at least 5% of ethyl cellulose, weight per weight of the entire formulation;
compressing said granulate into cores; and
coating said cores with a mixture of a water soluble cellulosic polymer selected from the group consisting of hydroxypropyl methylcellulose, hydroxypropyl cellulose, hydroxyethyl cellulose, methyl cellulose, and carboxymethyl cellulose;
wherein said granulate is prepared using a low shear granulator; and
wherein said coating is applied using a perforated pan coater.

31. A process for manufacturing a coated tablet containing venlafaxine, the process comprising:
preparing a granulate of venlafaxine, hydroxypropyl methylcellulose, microcrystalline cellulose and ethyl cellulose, wherein an amount of said hydroxypropyl methylcellulose is greater than about 8% weight per weight of the tablet, an amount of said microcrystalline cellulose is greater than about 40% weight per weight of the tablet, and an amount of said ethyl cellulose is greater than about 5% weight per weight of the tablet;
compressing said granulate into cores; and
coating said cores with a mixture of aqueous ethyl cellulose and hydroxypropyl methylcellulose to obtain the coated tablets;
wherein said granulate is prepared using a low shear granulator; and
wherein said coating is applied using a perforated pan coater.

32. The process of claim 1, wherein said core further comprises a lubricant.

33. The process of claim 32, wherein said lubricant is selected from the group consisting of stearate salts, stearic acid, talc, castor oil, hydrogenated palm oil, starch, polyethylene glycol, sodium stearyl fumarate, compritol, waxes, or a combination thereof.

34. The process of claim 33, wherein said lubricant comprises magnesium stearate.

35. The process of claim 34, wherein said magnesium stearate is present in an amount of from about 0.25% to about 5% weight per weight of the core.

36. The process of claim 35, wherein said magnesium stearate is present in an amount of up to about 2% weight per weight of the core.

37. The process of claim 1, wherein said core further comprises a plasticizer.

38. The process of claim 37, wherein said plasticizer is selected from the group consisting of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

39. The process of claim 38, wherein said plasticizer comprises dibutyl sebacate.

40. The process of claim 39, wherein said plasticizer further comprises polyethylene glycol.

41. The process of claim 38, wherein said plasticizer is present in an amount of up to about 5% weight per weight of the total formulation.

42. The process of claim 41, wherein said plasticizer is present in an amount of from about 0.01% to about 3% weight per weight of the total formulation.

43. A coated tablet containing a serotonin-norepinephrine reuptake inhibitor, obtained by a process comprising:
preparing a granulate of the serotonin-norepinephrine reuptake inhibitor using a low shear granulator;
compressing said granulate into a core; and
applying a coating to said core using a pan coater.

44. A coated tablet prepared according to the process of claim 1.
